(19) United States
(12) Patent Application Publication
(10) Pub. No.: US 2021/0213190 A1
(43) Pub. Date: Jul. 15, 2021

(54) PREDICTION DISPLAY SYSTEM AND TREATMENT METHOD

(71) Applicant: TERUMO KABUSHIKI KAISHA, Tokyo (JP)

(72) Inventor: Satoshi SAWADA, Kanagawa (JP)

(73) Assignee: TERUMO KABUSHIKI KAISHA, Tokyo (JP)

(21) Appl. No.: 17/144,729

(22) Filed: Jan. 8, 2021

(30) Foreign Application Priority Data
Jan. 9, 2020 (JP) 62-958777

(51) Int. Cl. A61M 1/36; G16H 50/30; G16H 20/40; A61M 60/30

(52) U.S. Cl. CPC A61M 1/367; G16H 50/30; G16H 20/40; A61M 60/30; A61M 1/3663; A61M 2205/502; A61M 60/279; A61M 2230/207; A61M 2230/208; A61M 2230/04; A61M 2230/50; A61M 2205/3365; A61M 2230/205

(57) ABSTRACT

To provide a prediction display system and the like that utilize information obtained before an operation and during the operation, and predict an onset risk of a postoperative complication such as an acute kidney injury which has become a particularly great clinical problem. A prediction display system is configured such that an onset risk of a complication occurring after an operation is predicted using at least one of pH (hydrogen ion exponent), pO2 (oxygen partial pressure), Ht (hematocrit value), Hb (hemoglobin), DO2 (transportation amount of oxygen), and pCO2 (arterial blood carbon dioxide partial pressure) as parameters of blood gas taken before the operation and during the operation, and the onset risk of the complication occurring after the operation is displayed.

### CROSS-REFERENCES TO RELATED APPLICATIONS

This application claims priority to Japanese Patent Application No. 2019-95877 filed on Jan. 9, 2020, the entire content of which is incorporated herein by reference.

### FIELD OF THE INVENTION

The present invention generally relates to a prediction display system and a treatment method that predict an onset risk of a postoperative complication(s) or the like such as an acute kidney injury.

### BACKGROUND DISCUSSION

Conventionally, it has been known that cardiac surgeries or the like accompany postoperative complications such as an acute kidney injury (AKI). These have created significant medical problems also from viewpoints of medical economics (medical costs or health economics) and patient QOL (Quality Of Life).

Accordingly, in order to predict occurrence of such a postoperative complication in advance, in the case of the AKI, for example, a plurality of biomarkers that are sensitive to a kidney injury and enable detection at an earlier stage than serum creatinine have recently been reported. An example of this is disclosed in Japanese Patent No. 4879993.

In Japan, NGAL (Neutrophil Gelatinase-Associated Lipocalin) and L-FABP (Liver-type Fatty Acid-Binding Protein) are listed in insurance, and globally, there are many reports on NGAL and IGFBP-7/TIMP-2 (Insulin-like Growth Factor Binding Protein-7/Tissue Inhibitor of Metalloproteinases-2). Both can detect nephropathy in a few hours to approximately 12 hours.

### SUMMARY

However, a difficulty remains in selecting patients to which the biomarkers and the like are applied, and the biomarkers and the like are relatively expensive. Also, when a postoperative complication such as an AKI is found, a change in procedure that should be implemented has not been established.

In addition, approximately one third of surgical patients develop a postoperative complication such as an AKI. Once a postoperative complication such as an AKI is developed, medical cost, the number of stay days, and the like are greatly increased, and a risk of a chronic renal failure leading to dialysis is reported to increase to nine times. Thus, preventing postoperative complication when the risk of developing postoperative complication is increased is very important.

On the other hand, though a guideline defining the AKI has made transitions to RIFLE (Risk/Injury/Failure/Loss/End-stage), AKIN (Acute Kidney Injury Network), and KDIGO (Kidney Disease: Improving Global Outcomes), each is defined by changes in urinary volume and serum creatinine (sCr) over a specific period.

However, changes in urinary volume such as oliguria do not occur along with a kidney injury in all cases. In addition, there is a time lag such that serum creatinine increases one to two days after the kidney is damaged. Thus, a preventive intervention is difficult after an increase in serum creatinine is found.

A prediction display system and a treatment method disclosed here utilize information obtained before an operation and during the operation, and predict the onset risk of a postoperative complication such as an acute kidney injury which has become a particularly great clinical problem.

A prediction display system comprises a controller that that includes a control section that predicts an onset risk of a complication occurring after a surgical operation using at least one of pH (hydrogen ion exponent), pO2 (oxygen partial pressure), Ht (hematocrit value), Hb (hemoglobin), DO2 (transportation amount of oxygen), and pCO2 (arterial blood carbon dioxide partial pressure) as parameters of blood gas taken before the surgical operation and during the surgical operation; and a display that is connected to the control section and that displays the onset risk of the complication occurring after the surgical operation.

Preferably, in the prediction display system, the onset risk of the complication after the operation is predicted using at least one of pH, pO2, Ht, Hb, DO2, and pCO2 as the parameters of the blood gas in data obtained during operation of an extracorporeal circulation apparatus and at least one of a pump flow rate, a pump rotational speed, a temperature, a blood circulation time, and an aorta interruption time as operating conditions of the extracorporeal circulation apparatus, and the onset risk of the complication after the operation is displayed.

Preferably, in the prediction display system, the complication is an acute kidney injury occurring after the operation.

Preferably, in the prediction display system, the onset risk of the acute kidney injury after the operation is predicted using at least one of an arterial pressure, a heart rate, an ECG (electrocardiogram), a urinary volume, a peripheral oxygen saturation, and a deep body temperature as a temperature within a body as vital data, and the onset risk of the acute kidney injury after the operation is displayed.

Preferably, in the prediction display system, the onset risk of the complication is an onset risk of at least one of a low cardiac output syndrome (LOS), an acute kidney injury (AKI), a cerebral infarction, a mediastinitis, and an acute respiratory distress syndrome (ARDS) as the complication after the operation.

Preferably, in the prediction display system, a prediction is performed using the onset risk, and an extracorporeal circulation condition of the extracorporeal circulation apparatus, the extracorporeal circulation condition being likely to reduce the onset risk of the complication after the operation, is analyzed and displayed.

Preferably, in the prediction display system, a change in flow rate and rotational speed of either a blood removing pump or a blood sending pump of the extracorporeal circulation apparatus is suggested.

Preferably, in the prediction display system, whether or not there is a need for a hemoconcentrator circuit of the extracorporeal circulation apparatus is determined and displayed on the basis of the onset risk of the complication after the operation.

Preferably, in the prediction display system, the extracorporeal circulation condition is automatically changed on the basis of the prediction.

Preferably, in the prediction display system, an administration system for reducing the onset risk of the complication after the operation is controlled on the basis of the prediction.

Preferably, in the prediction display system, the onset risk of the complication is predicted using at least one of an age, a sex, an address, a race, a family structure, an anamnesis, and a preoperative test value that affect a prognosis as characteristics of a patient, and the onset risk of the complication is displayed.

Preferably, in the prediction display system, the onset risk of the complication is predicted using at least one of an operative method, a lesion site, an operation time, an operator, an anesthesiologist, a dosage, a kind of medicine, an anesthesia induction time, and presence or absence of a blood transfusion as information regarding the operation, and the onset risk of the complication is displayed.

According to another aspect, a prediction display system comprises a controller that that includes a control section that predicts an index of at least one of the number of ICU (intensive care unit) stay days, presence or absence of in-hospital death, and presence or absence of continuous renal replacement therapy (CRRT) introduction related to medical economies using: i) at least one of pH, pO2, Ht, Hb, DO2, and pCO2 as parameters of blood gas in data obtained during operation of an extracorporeal circulation apparatus; and ii) at least one of a pump flow rate, a pump rotational speed, a temperature, a blood circulation time, and an aorta interruption time as operating conditions of the extracorporeal circulation apparatus; and a display that is connected to the control section and that displays the predicted index.

Preferably, in the prediction display system, an onset risk of a main complication and another management item are taken on an axis of ordinate and an axis of abscissa, and are two-dimensionally displayed on a matrix table, and a region to which a transition is made in the matrix table during an ICU stay is predicted and displayed.

Preferably, the matrix table of the prediction display system two-dimensionally displays a group of high onset risks of the main complication and a group of low onset risks of the main complication, and a group of high probabilities of the number of ICU stay days (group of patients likely to have a relatively long ICU stay following surgery), in-hospital death, and CRRT introduction related to medical economics (medical costs or health economics) and a group of low probabilities of the number of ICU stay days (group of patients likely to have a relatively short ICU stay following surgery), in-hospital death, and CRRT introduction related to medical economics, and the region to which a transition is made in the matrix table during the ICU stay is predicted and displayed.

Preferably, in the prediction display system, the matrix table has a group of high onset risks of the main complication and a group of low onset risks of the main complication, and a group of high risks of occurrence of a symptom that needs an action such as an edema or an infection and a group of low risks of occurrence of the symptom, and the region to which a transition is made two-dimensionally in the matrix table is predicted and displayed.

Another aspect pertains to a treatment method that involves use of an extracorporeal circulation apparatus that circulates a patient's blood outside the patient during a surgical operation on the patient. The treatment method comprises: predicting an onset risk of a complication using: i) at least one of pH, pO2, Ht, Hb, DO2, and pCO2 as parameters of blood gas in data obtained during operation of the extracorporeal circulation apparatus; and ii) at least one of a pump flow rate, a pump rotational speed, a temperature, a blood circulation time, and an aorta interruption time as operating conditions of the extracorporeal circulation apparatus to obtain a predicted onset risk of the complication; displaying the predicted onset risk of the complication on a screen; and optimizing a circulation condition of the extracorporeal circulation apparatus based on the predicted onset risk of the complication being displayed on the screen.

The prediction display system and treatment method are advantageous in that they utilize information obtained before a surgical operation (medical procedure) and during the surgical operation, and predict the onset risk of a postoperative complication such as an acute kidney injury which has become a particularly great clinical problem.

### BRIEF DESCRIPTION OF THE DRAWINGS

FIG. 1 is a schematic explanatory diagram illustrating a main configuration of an extracorporeal circulation apparatus 10 used in a prediction display system according to an embodiment disclosed by way of example.

FIG. 2 is a schematic block diagram illustrating a main configuration of a controller 40 and other aspects of the prediction display system in FIG. 1.

FIG. 3 is a schematic flowchart illustrating an example of operation of the prediction display system according to one embodiment.

FIG. 4 is an example of a manner of calculating a medical cost as an example of medical economics.

FIG. 5 is a diagram illustrating data as a basis of the medical cost.

FIG. 6 illustrates a matrix table of an AKI onset risk as an example of the onset risk of a postoperative complication and a medical cost as an example of medical economics or the like.

FIG. 7 illustrates a matrix table of the AKI onset risk and an edema.

### DETAILED DESCRIPTION

Set forth below with reference to the accompanying drawings is a detailed description of embodiments of a prediction display system and a treatment method representing examples of the inventive prediction display system and treatment method disclosed here.

It is to be noted that while embodiments to be described below are preferred specific examples of the prediction display system and treatment method, and therefore technically preferable various limitations are attached thereto, the scope of the present invention is not limited to these aspects unless the following description so states.

### First Embodiment

(Extracorporeal Circulation Apparatus 10)

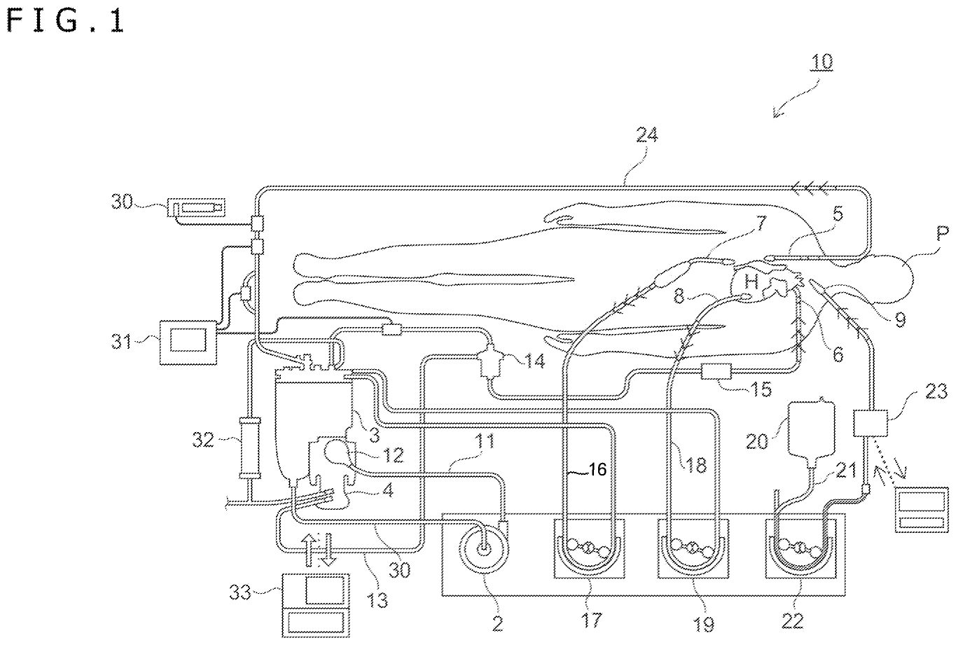

FIG. 1 is a schematic explanatory diagram illustrating a main configuration of an extracorporeal circulation apparatus 10 used in a prediction display system as one embodiment disclosed here.

The extracorporeal circulation apparatus 10 illustrated in FIG. 1 performs extracorporeal circulation operations. In the extracorporeal circulation operation, the extracorporeal circulation apparatus 10 performs blood circulation operation and gas exchange operation (oxygen addition and/or carbon dioxide removal) for blood in a case where blood circulation in a heart is temporarily stopped by a cardiac surgery, for example.

In a case where a surgery on a heart H of a patient P is performed, for example, the extracorporeal circulation apparatus 10 illustrated in FIG. 1 actuates a blood sending pump 2 as a centrifugal pump of the extracorporeal circulation apparatus 10, and thereby removes blood from the patient P and guides the removed blood to a reservoir 3.

Then, the blood retained in the reservoir 3 is sent to an oxygenator 4, for example, as an oxygenator unit. Thereafter, the blood is oxygenated by performing a gas exchange in the blood by the oxygenator 4. The oxygenated blood is then returned to the patient P. An oxygenator extracorporeal blood circulation can be thereby performed.

Thus, the extracorporeal circulation apparatus 10 is a device that substitutes for the heart and lungs.

As illustrated in FIG. 1, a blood removing cannula 5, a blood sending cannula 6, a suction cannula 7, a vent cannula 8, and a cardioplegia cannula 9 are arranged in the patient P.

In the following, description will be made in order of a circulation circuit from the blood removing cannula 5 to the blood sending cannula 6, a circuit from the suction cannula 7 to the reservoir 3, a circuit from the vent cannula 8 to the reservoir 3, a circuit from a cardioplegic solution bag 20 to the cardioplegia cannula 9, and the like.

(Circulation Circuit from Blood Removing Cannula 5 to Blood Sending Cannula 6)

As illustrated in FIG. 1, the blood removed via the blood removing cannula 5 inserted and placed in a superior vena cava and an inferior vena cava of the patient P is guided to the reservoir 3 through a blood removing tube 24.

In addition, as illustrated in FIG. 1, an oxygen saturation monitor 30 and a blood gas monitor 31 are formed on or provided along the blood removing tube 24.

The blood temporarily retained in the reservoir 3 is guided to the blood sending pump 2 via a first connecting tube 30 in FIG. 1, and is further guided to the oxygenator 4 via a second connecting tube 11, a heat exchanger 12, and the like.

As described above, the blood guided to the oxygenator 4 is oxygenated by performing a gas exchange in the blood by the oxygenator 4, and is thereafter returned to inside of the body of the patient P from the blood sending cannula 6 via a solution feeding tube 13.

Arranged on or along the solution feeding tube 13 are, for example, an artery filter 14 for removing air bubbles in the solution feeding tube 13, and an air bubble detector 15 for detecting air bubbles in the blood.

The reservoir 3 disposed on this circulation circuit is a container that internally has a storage space (storage chamber). The reservoir 3 is configured to temporarily retain, for example, the blood removed from the blood removing cannula 5, the blood emitted during an operation and guided from the suction cannula 7 as will be described later, the blood from the vent cannula 8 extracting air from a left ventricle, an aortic root, as will be described later.

Because the reservoir 3 thus temporarily retains the blood, the blood removed from the patient P is returned to the patient P without excess or insufficiency.

In addition, the blood sending pump 2 of the circulation circuit is driven by a motor or the like. When the blood sending pump 2 is driven, the blood is sent from the blood removing cannula 5 to the blood sending cannula 6 via the reservoir 3, the oxygenator 4, and the like.

In addition, the oxygenator 4 of the circulation circuit performs a gas exchange (oxygen addition and/or carbon dioxide removal) for the blood, as described above. The oxygenator 4 is, for example, a membrane oxygenator, and is preferably a hollow fiber membrane oxygenator. The oxygenator 4 is supplied with an oxygen gas from an oxygen gas supply unit.

(Circuit from Suction Cannula 7 to Reservoir 3)

The blood emitted during the operation is guided from the suction cannula 7 illustrated in FIG. 1 to the reservoir 3 via a suction cannula tube 16, and is retained in the reservoir 3.

A suction pump (blood removing pump) 17 is disposed on or along the circuit. The blood is guided to the reservoir 3 by operation of the suction pump 17.

(Circuit from Vent Cannula 8 to Reservoir 3)

The vent cannula 8 for extracting air from the left ventricle, the aortic root, and the like is guided to the reservoir 3 via a vent cannula tube 18.

A vent pump 19 is disposed on or along this circuit. The air, the blood, and the like are guided to the reservoir 3 by an operation of the vent pump 19.

(Circuit from Cardioplegic Solution Bag 20 to Cardioplegia Cannula 9)

A cardioplegic solution is stored in the cardioplegic solution bag 20, and is administered into the patient P via a cardioplegic solution tube 21 and the cardioplegia cannula 9.

A cardioplegic solution pump 22 is disposed on or along this circuit. The cardioplegic solution is sent to the patient P side by operation of the cardioplegic solution pump 22. A pump for cardioplegic solution may be different from a pump for circulation.

A heat exchanger 23 is disposed on or along the cardioplegic solution tube 21.

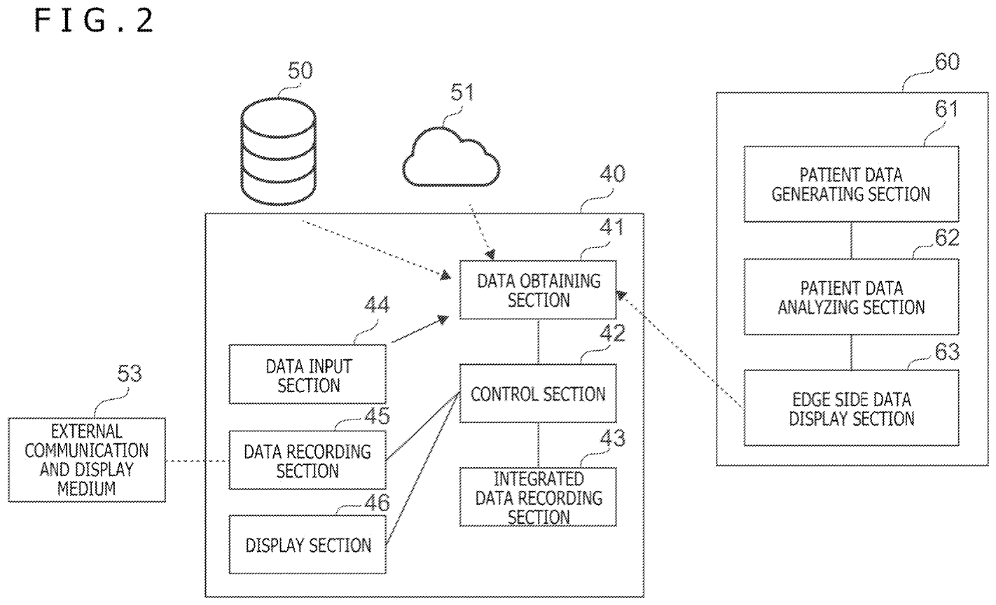

In addition, as illustrated in FIG. 1, the extracorporeal circulation apparatus 10 has a hemoconcentrator 32, a water cooling and heating controller 33, and the like, and also includes a controller 40 illustrated in FIG. 2 for performing control of the whole of the extracorporeal circulation apparatus 10 and the like.

FIG. 2 is a schematic block diagram illustrating a main configuration of the controller 40 in FIG. 1 and the like.

As illustrated in FIG. 2, the controller 40 includes a data obtaining section 41.

As illustrated in FIG. 2, the data obtaining section 41 obtains various kinds of data from an EHR (Electronic Health Record) server 50 possessed by a hospital or the like and a cloud 51.

The EHR server 50 is an electronic health record server. Medical data of patients and the like is recorded on the EHR server 50.

In addition, the EHR server may be a data warehouse (DWH).

As illustrated in FIG. 2, the controller 40 includes, for example, a control section 42, and is configured to control the data obtaining section 41, an integrated data recording section 43, a data input section 44, a data recording section 45, a display section or display 46.

In addition, as illustrated in FIG. 2, the data recording section 45 of the controller 40 may display contents recorded therein on not only the display section 46 but also an external communication and display medium 53 or the like.

The external communication and display medium 53 includes, for example, the oxygen saturation monitor 30 and the blood gas monitor 31 in FIG. 1.

Communication may be performed not only in a wired manner but also in a wireless manner.

Further, the data obtaining section 41 of the controller 40 may obtain patient data or the like from a vital data generating device 60 illustrated in FIG. 2.

As illustrated in FIG. 2, the vital data generating device 60 includes a patient data generating section 61, a patient data analyzing section 62, and an edge side data display section 63.

Various kinds of devices such as the controller 40 illustrated in FIG. 1 and FIG. 2, for example, include a computer or the like, that is, include a CPU (Central Processing Unit), a RAM (Random Access Memory), a ROM (Read Only Memory), and the like not illustrated, which are connected to each other via a bus.

When data that needs a certain processing speed is handled, a device having provision therefor is preferable, and a GPU (Graphics Processing Unit), a KPU (Kernel Processing Unit), or the like, for example, may be included.

(Prediction Timing of Prediction Display System and the Like)

A prediction made by the prediction display system according to one embodiment is possible at any time before the operation, during the operation, and after the operation.

Specifically, cases before the operation include not only a case where a prediction is triggered by timing of input of various kinds of data such as vital data of the patient P but also a case where a prediction is performed when data input is completed and a prediction button of the controller 40 or the like is pressed.

In addition, cases during the operation include a case where a prediction is performed in real time according to the vital data of the patient which vital data changes in real time, a case where a prediction is performed at intervals of a certain unit time moving average, a case where a prediction is triggered by timing of data input, and the like.

Then, cases after the operation include, for example, a prediction after processing of data of the patient before the operation and during the operation or the like into a format that can be input into a “learned algorithm.”

(Example of Operation of Prediction Display System According to Present Embodiment)

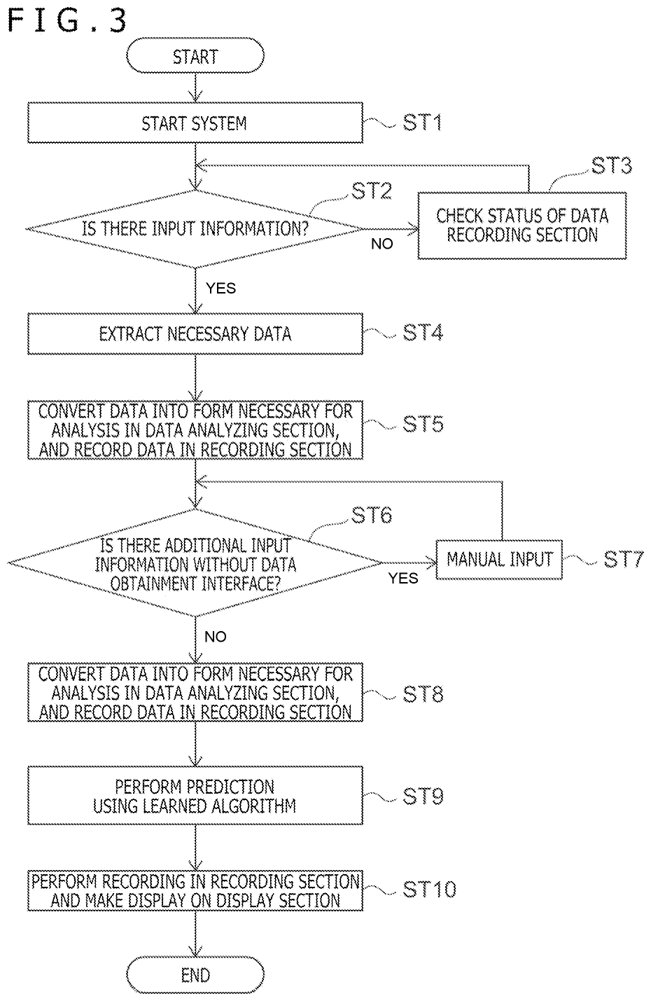

FIG. 3 is a flowchart illustrating an example of operation of the prediction display system according to the present embodiment.

The flowchart represents an example of making a predictive display of a postoperative complication using preoperative and intraoperative data during the operation on the patient P. Operation and the like will be described in the following by using such an example.

In the present embodiment, the “postoperative complication” includes an acute kidney injury (AKI), a low cardiac output syndrome (LOS), a cerebral infarction, a mediastinitis, an acute respiratory distress syndrome (ARDS), and the like. In the case of the AKI, an onset probability in each AKI stage and an onset probability of the AKI after entry into an ICU are predicted.

First, the extracorporeal circulation apparatus 10 having the present system is used when the patient P undergoes an operation on the heart, for example.

Then, a person in charge of medical information such as a doctor uses the present system to predict a complication such as an AKI after the operation.

First, the system is started in step (hereinafter referred to as “ST”) 1 in FIG. 3.

Next, the processing proceeds to ST2. In ST2, the system determines whether or not information is input to the controller 40.

When no information is input in ST2, the processing proceeds to ST3.

In ST3, a status of the data recording section 45 of the controller 40 in FIG. 2 is checked.

When information is input in ST2, on the other hand, the processing proceeds to ST4.

In ST4, data necessary for a prediction of a postoperative complication is extracted from the data recording section 45 of the controller 40.

Here, the necessary data is, specifically, preoperative data and intraoperative data.

Accordingly, the preoperative data and the intraoperative data will be described in the following.

(Preoperative Data)

The preoperative data includes patient background, preoperative kidney functions, a blood test result, presence or absence of diabetes, anamnesis information, and the like recorded in, for example, the EHR server 50.

In addition, the preoperative data includes an operative method, a lesion site, an operator, and an anesthesiologist as information regarding the operation.

(Intraoperative Data)

The intraoperative data is data obtained mainly from the extracorporeal circulation apparatus 10. The intraoperative data includes, for example, vital information, operation information, a peripheral tissue enzyme level” blood circulation state information, and heart-lung machine (CPB) circulation state information of the extracorporeal circulation apparatus.

Each piece of information will be described in the following by citing a specific example.

Examples of the vital information includes BP (blood pressure), HR (heart rate), ECG (electrocardiogram), a urinary volume, SpO2 (oxygen saturation), an arterial pressure, a peripheral oxygen saturation, a deep body temperature, and the like.

The operation information includes an operation time, a CPB (heart-lung machine) driving time, presence or absence of aorta interruption, a dosage, a kind of medicine, an anesthesia induction time, presence or absence of a blood transfusion, and the like.

The peripheral tissue oxygen level includes rSO2 (tissue oxygen saturation), SpO2 (oxygen saturation), a pulse rate, and the like.

The blood circulation state information includes pH (hydrogen ion exponent), pO2 (oxygen partial pressure), Ht (hydrogen ion exponent), Hb (hemoglobin), DO2 (oxygen delivery), pCO2 (carbon dioxide partial pressure), Temp (temperature), and the like.

The heart-lung machine (CPB) circulation state information of the extracorporeal circulation apparatus includes Temp (temperature), a pump rotational speed, a pump flow rate, an aorta interruption time, a blood circulation time, and the like.

In addition, each of these pieces of data may be used as it is as raw data. Alternatively, a time change graph of all of these parameters may be displayed on the edge side data display section 63 of the vital data generating device 60 in FIG. 2 or the like, a screen thereof may be obtained, and the screen may be set as an input factor. Further, an input factor corresponding to a complication may be set in advance.

Next, the processing proceeds to ST5. In ST5, the data is converted into a form necessary for analysis in the data analyzing section, and is recorded in the data recording section 45.

Next, the processing proceeds to ST6, where the system determines whether or not there is additional input information without a data obtainment interface (i.e., additional input information obtained by a machine not connected to data storage (e.g., EHR) or not in-line).

When it is determined in ST6 that there is additional input information, the processing proceeds to ST7.

In ST7, necessary data is input manually.

Next, in ST8, the data is converted into a form necessary for analysis in the data analyzing section, and is recorded in the data recording section 45.

Next, the processing proceeds to ST9. In ST9, a prediction is performed by using a learned algorithm.

Machine learning is performed in the step in which a prediction is performed by using the learned algorithm.

The machine learning is classified as supervised learning. The machine learning is performed in two separate stages of learning and recognition and prediction.

In the learning stage, a data set is created, a rule and a pattern of the data are learned on the basis of the data set, and a learned algorithm is created.

Then, the recognition and prediction of the machine learning are performed on the basis of the learned algorithm.

Next, the processing proceeds to ST10. In ST10, a prediction result is recorded in the data recording section 45, and is displayed on the display section 46.

As a result of the above, predictive information on the onset risk of a postoperative complication is notified to a medical worker such as the doctor during the operation.

(Predictive Display of Onset Risk of Complication)

Predictive display of the onset risk of a complication in the present embodiment includes, for example, predicting a set value that reduces the onset risk of a complication, and displaying the set value on the display section 46.

In addition, in the present embodiment, the onset risk of the complication in a case where the set value is changed to a certain specified value may be displayed at the same time. The prediction display system may thus display the risk of a complication, predict a set value (e.g., involving perfusion machine intervention or monitoring vital parameters) that should lead to a reduction in the risk of complication, displaying that predicted set value which should lead to a complication risk reduction, and displaying the reduced risk of complication at the predicted set value.

More specifically, when information indicating a decrease in BP (blood pressure) as vital information is obtained during the operation, a prediction that a cerebral infarction risk increases when the BP (blood pressure) is the same after two minutes is made by the learned algorithm on the basis of a set value, a specified value, or the like, and the prediction (cerebral infarction risk increases) is displayed.

In addition, a “start of vasopressor administration” or the like may also be displayed as a medical action for improvement.

In addition, as another example, when information indicating a decrease in Ht (hematocrit value) is obtained during the operation, a prediction that an AKI risk is decreased when water content is reduced is made by the learned algorithm, and the prediction (AKI risk is decreased when water content is reduced) is displayed.

In addition, “opening of a hemoconcentrator circuit,” “preparation of a diuretic,” or the like may also be displayed as a medical action for improvement.

In addition, in the present embodiment, the onset risk of a complication may be predicted using at least one of an age, a sex, an address, a race, a family structure, an anamnesis, and a preoperative test value that affect a prognosis as characteristics of the patient, and the onset risk of the complication may be displayed.

Further, the onset risk of a complication may be predicted using at least one of an operative method, a lesion site, an operation time, an operator, an anesthesiologist, a dosage, a kind of medicine, an anesthesia induction time, presence or absence of a blood transfusion, and the like as information regarding the operation, and the onset risk of the complication may be displayed.

(Modification of First Embodiment)

Much of a configuration according to a present modification is similar to that of the foregoing first embodiment, and so a detailed description of features and aspects already described above will not be repeated. And in the description below, features that are the same or similar to those described above are identified by the same reference numerals. The following description will primarily focus on differences relative to the embodiment described above.

The foregoing first embodiment involves predicting and displaying the onset risk of a postoperative complication or the like and further displaying at the same time a medical action for improvement. The present modification further automatically sets and changes a circulation condition (operating condition) of the extracorporeal circulation apparatus 10 on the basis of the prediction of the onset risk of the postoperative complication or the like.

For example, a pump and an analyzing unit are linked to each other, and cooperation with an infusion pump or a syringe pump, feedback on administration, and the like are performed.

In addition, the flow rate and rotational speed of one of the suction pump (blood removing pump) 17 and the blood sending pump 2 of the extracorporeal circulation apparatus 10 may be changed based on the prediction of the onset risk of the postoperative complication, and the presence or absence of the hemoconcentrator circuit of the extracorporeal circulation apparatus 10 may be determined, displayed, and controlled on the basis of the prediction of the onset risk of the postoperative complication.

Further, the extracorporeal circulation condition of the extracorporeal circulation apparatus 10 may be automatically changed on the basis of the prediction, and an administration system for reducing the onset risk of the postoperative complication may be controlled on the basis of the prediction.

A specific example is as follows.

When information indicating a decrease in BP (blood pressure) as vital information is obtained during the operation, a prediction that a cerebral infarction risk is increased when the BP (blood pressure) is the same after two minutes is made by the learned algorithm on the basis of a set value, a specified value, or the like, the circulation condition of the extracorporeal circulation apparatus 10 is changed, and a start of vasopressor administration or the like is performed. In other words, during an operation, if information about a decrease in blood pressure is obtained or received, the learned algorithm predicts (based on a set value, a specified value or the like) that there is an increased cerebral infarction risk when the blood pressure decrease remains after two minutes, whereupon the circulation condition of the extracorporeal circulation apparatus is changed and vasopressor administration or the like is initiated.

At this time, a step of confirming an intention of the anesthesiologist may be added. In this case, vasopressor administration is actually started when a displayed instruction is accepted (i.e., when an anesthesiologist accepts or agrees that vasopressor administration should be initiated).

In addition, as another example, when information indicating a decrease in Ht (hematocrit value) is obtained or received during the operation, a prediction that an AKI risk is decreased when water content is reduced is made by the learned algorithm, the circulation condition of the extracorporeal circulation apparatus is changed, and opening of a hemoconcentrator circuit, preparation of a diuretic, or the like is performed.

Thus, according to the present modification, automatic operation of the heart-lung machine as the extracorporeal circulation apparatus 10 that performs parameter control with even the onset risk of a postoperative complication taken into consideration can be performed.

In addition, while the present embodiment is configured to display predictive information or operate the extracorporeal circulation apparatus 10 according to the predictive information, the present invention is not limited to this, but includes a medical action of optimizing the circulation condition of the extracorporeal circulation apparatus 10 according to the predictive information, the medical action being performed by the doctor.

### Second Embodiment

Much of the configuration of the apparatus or system according to this second embodiment is similar to that of the foregoing first embodiment and modification, and so a detailed description of features and aspects already described above will not be repeated. In the description below, features that are the same or similar to those described above are identified by the same reference numerals. The following description will primarily focus on differences relative to the embodiment and modification described above

Unlike the foregoing first embodiment, the present embodiment is configured to predict an index related to medical economics or medical costs using the step of the learned algorithm.

Specifically, a prediction of the number of ICU stay days, a prediction of a death probability, and the like are performed by the learned algorithm on the basis of the preoperative data and the intraoperative data described above or the like, and these predictions are stored as an index related to medical economics or medical costs.

In addition, this embodiment is configured to predict whether a medical action or medical intervention related to cost such as an extension of the number of ICU stay days or CRRT (continuous renal replacement therapy) will occur in the future by using the step of the learned algorithm on the basis of the preoperative data and the intraoperative data described above or the like.

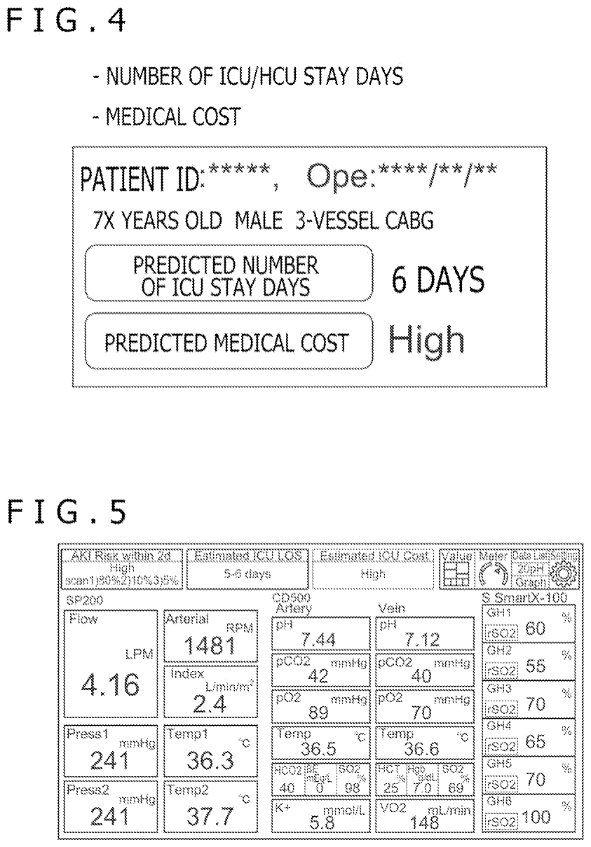

FIG. 4 is an example of calculating or predicting a medical cost (e.g., high cost) as an example of medical economics (medical costs or health economics). FIG. 5 is a diagram illustrating data as a basis of the medical cost.

The present embodiment generates a two-dimensional prediction of a position of a risk on a matrix table combining information regarding the index related to medical economics or a prediction of a possibility that a medical action related to cost will occur in the future in FIG. 4 or the like with the prediction of the onset risk of a postoperative complication in the foregoing first embodiment, and displays the two-dimensional prediction on the display section 46.

A medical worker such as a doctor can thereby recognize these relations at a glance.

That is, the matrix table can two-dimensionally display a group of high onset risks of a main complication and a group of low onset risks of the main complication, and a group of high probabilities of the number of ICU stay days, in-hospital death, CRRT introduction related to medical economics (medical costs or health economics)and a group of low probabilities of the number of ICU stay days, in-hospital death, CRRT introduction related to medical economics (medical costs or health economics), and predict and display a region to which a transition is made in the matrix table during an ICU stay.

Here, the high groups and low groups are divided as follows, for example.

For example, the group of high onset risks of the complication and the group of low onset risks of the complication are divided according to the presence or absence of an onset of the complication within, for example, seven days, and the number of ICU stay days is divided according to whether or not a stay in the ICU for three days or more is made, for example.

In addition, the in-hospital death is divided according to whether or not the in-hospital death is a death within seven days.

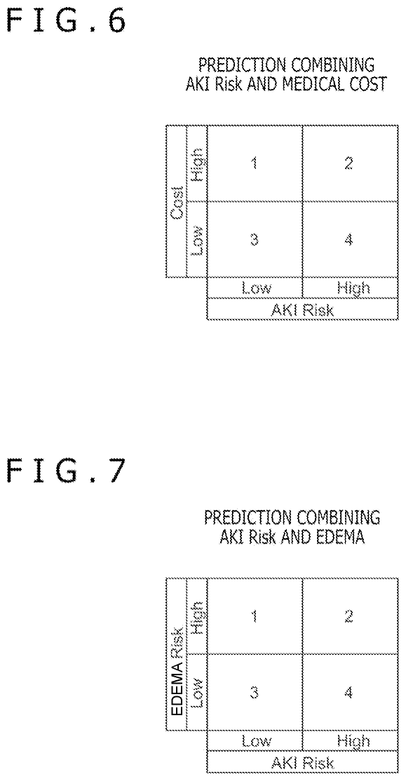

FIG. 6 illustrates a matrix table of an AKI onset risk as an example of the onset risk of a postoperative complication and medical costs as an example of medical economics (medical costs or health economics)or the like. FIG. 7 illustrates a matrix table of the AKI onset risk and an edema, wherein edema is a representative symptom or an example of a symptom.

As illustrated in FIG. 6 and FIG. 7, by using such matrix tables, it is possible to two-dimensionally display a group of high onset risks of a main complication and a group of low onset risks of the main complication, and a group of high risks of occurrence of a symptom that needs an action such as an edema or an infection and a group of low risks of occurrence of the symptom, and predict and display a region to which a transition is made. For example, medical personnel might confirm that a patient's circumstances evidence a high AKI risk and a high medical cost (region 2) in FIG. 6, thus conveying a need to intervene to reduce the risk and/or the medical cost. The system may predict changes in risk and medical cost if certain actions are undertaken in a way resulting in the patient's AKI risk and medical cost moving to a different region (for example region 3) in FIG. 6. On the other hand, if medical personnel confirm that a patient's circumstances represent, for example, a low AKI risk and a low medical cost (region 3) or perhaps a high AKI risk and a low medical cost (region 4), there may not be a need for current intervention. But if the medical personnel see the patient's status transition on the two-dimensional display (for example moving from region 3 to region 2 in FIG. 6), considered at various times such as preoperation/intraoperation/postoperation, medical personnel can know the status of the transition of the patient, and the medical personnel can treat the patient in advance to avoid the onset of a complication. The mapping of a patient's status transition can include not only real value but predicted value like predicted status after intervention based on the intervention supposed or suggested by the algorithm. Also, as noted, the prediction display system may predict and display a region to which a transition is made. That is, a patient's status and status transition may be displayed as a marker(s) on the two-dimensional display in FIG. 6. For example, a marker(s) indicating a predicted status may be different from a marker(s) indicating a past status so that a medical person can understand easily. And, when a user moves a cursor over a status marker, information such as time, situation, memo, operator may be displayed (i.e., pop-up) to identify a status pointed to by the cursor.

In addition, in the case of FIG. 6, when the AKI risk is “high,” and the medical cost is “high,” divisions can also be further made and displayed according to costs incurred for three and seven days from hospitalization or the like.

In addition, while the present embodiment displays the two-dimensional matrix tables of four events, the present invention is not limited to this, but when in-hospital death is taken on an axis of the abscissa, for example, 2×3 events including survival and deaths within 7 days and 30 days, and the like can be adopted. The present invention is thus not limited to four events.

As described above, according to the foregoing embodiment, high risk patients can be stratified in advance by using data obtained during the operation, and a cardiac surgery or an extracorporeal circulation procedure with attention paid to a prognosis indicating that an AKI or the like is less likely to be caused is made possible. Thus, even when the doctor or the like performs the same procedure, a useful determination can be made from patient QOL and medical economics (medical costs or health economics).

In the foregoing embodiment, the learned algorithm used for prediction is constructed from data obtained in advance. In addition, the data obtained in advance includes data obtained from electronic medical records or the like recorded in the EHR server 50 or the like.

When the learned algorithm is constructed, the learned algorithm is prepared by a method such as regression analysis, machine learning, or a neural network (NN) using preoperative data and intraoperative data in the past.

The preoperative data includes, for example, the above-described operative method and the like. The intraoperative data includes, for example, the above-described vital information and the like. An algorithm construction can be performed by using the data associated with data on the postoperative complication.

The construction of the algorithm may use not only ordinary linear regression analysis, logistic regression analysis, SVM (Support Vector Machine), a random forest, a decision tree, and the like, but also ensemble learning using a plurality of models.

For example, LightGBM, Gradient Boosting, and the like correspond to this.

These methods are constantly updated to highly accurate methods, and therefore a method providing more accuracy may be selected.

In a neural network, it suffices to use a method such as a Conventional Neural Network (CNN), a Recurrent Neural Network (RNN), or Generative Adversarial Networks (GAN) according to a kind of data and the format of data obtained.

The learned algorithm may be not only constructed from data obtained in advance, but also configured to record data obtained in real time in an electronic medical record or the like and thereby update data in the electronic medical record or the like.

In that case, a prediction can be performed while original data necessary for algorithm construction is updated in an algorithm generating section cooperating with the data obtaining section 41, and an update is performed in conformity with a present situation and in real time.

As for an index of accuracy in that case, a confusion matrix (Confusion Matrix) that makes an evaluation on the basis of sensitivity and specificity is performed, AUROC (Area Under Receiver Operating Curve) may be used, and an accuracy rate (Accuracy), precision (Precision), reproducibility (Recall), an F-value (F1 score), and the like are used.

This may be automatically performed within a “prediction system,” or a selection may be allowed to be made as to which model to use each time the original data is updated.

In addition, because the accuracy of these models greatly affects a treatment result of the patient and a prognosis, there may be a configuration that performs external control via communication in addition to a selection within the hospital or the like.

The detailed description above describes embodiments and modifications of a prediction display system (post-operative medical condition prediction display system) and a treatment method representing examples of the inventive prediction display system and treatment method disclosed here. The invention is not limited, however, to the precise embodiments, modifications and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

### DESCRIPTION OF REFERENCE SYMBOLS

2: Blood sending pump
3: Reservoir
4: Oxygenator
5: Blood removing cannula
6: Blood sending cannula
7: Suction cannula
8: Vent cannula
9: Cardioplegia cannula
10: Extracorporeal circulation apparatus
11: Second connecting tube
12: Heat exchanger
13: Solution feeding tube
14: Artery filter
15: Air bubble detector
16: Suction cannula tube
17: Suction pump
18: Vent cannula tube
19: Vent pump
20: Cardioplegic solution bag
21: Cardioplegic solution tube
22: Cardioplegic solution pump
23: Heat exchanger
24: Blood removing tube
30: First connecting tube
40: Controller
41: Data obtaining section
42: Control section
43: Integrated data recording section
44: Data input section
45: Data recording section
46: Display section
50: EHR server
51: Cloud
53: External communication and display medium
60: Vital data generating device
61: Patient data generating section
62: Patient data analyzing section
63: Edge side data display section

What is claimed is:

1. A treatment method that involves use of an extracorporeal circulation apparatus that circulates a patient's blood outside the patient during a surgical operation on the patient, the treatment method comprising:
predicting an onset risk of a complication using: i) at least one of pH, pO2, Ht, Hb, DO2, and pCO2 as parameters of blood gas in data obtained during operation of the extracorporeal circulation apparatus; and ii) at least one of a pump flow rate, a pump rotational speed, a temperature, a blood circulation time, and an aorta interruption time as operating conditions of the extracorporeal circulation apparatus to obtain a predicted onset risk of the complication;
displaying the predicted onset risk of the complication on a screen; and
optimizing a circulation condition of the extracorporeal circulation apparatus based on the predicted onset risk of the complication being displayed on the screen.

2. The treatment method according to claim 1, wherein the complication is an acute kidney injury occurring after the surgical operation.

3. The treatment method according to claim 1, wherein the optimizing of the circulation condition of the extracorporeal circulation apparatus occurs automatically based on the predicted onset risk of the complication being displayed on the screen.

4. The treatment method according to claim 1, wherein the predicting of the onset risk of the complication is performed using a learned algorithm.

5. A prediction display system, comprising:
a controller that that includes a control section that predicts an onset risk of a complication occurring after a surgical operation using at least one of pH (hydrogen ion exponent), pO2 (oxygen partial pressure), Ht (hematocrit value), Hb (hemoglobin), DO2 (transportation amount of oxygen), and pCO2 (arterial blood carbon dioxide partial pressure) as parameters of blood gas taken before the surgical operation and during the surgical operation; and
a display that is connected to the control section and that displays the onset risk of the complication occurring after the surgical operation.

6. The prediction display system according to claim 5, wherein the control section is configured to predict the onset risk of the complication after the surgical operation using at least one of pH, pO2, Ht, Hb, DO2, and pCO2 as the parameters of the blood gas in data obtained during operation of an extracorporeal circulation apparatus and at least one of a pump flow rate, a pump rotational speed, a temperature, a blood circulation time, and an aorta interruption time as operating conditions of the extracorporeal circulation apparatus, and the display displays the predicted onset risk of the complication after the operation.

7. The prediction display system according to claim 5, wherein the complication is an acute kidney injury occurring after the surgical operation.

8. The prediction display system according to claim 5, wherein the control section is configured to predict the onset risk of an acute kidney injury after the surgical operation using at least one of an arterial pressure, a heart rate, an ECG (electrocardiogram), a urinary volume, a peripheral oxygen saturation, and a deep body temperature as a temperature within a body as vital data, and the display displays the predicted onset risk of the acute kidney injury after the surgical operation.

9. The prediction display system according to claim 5, wherein the onset risk of the complication is an onset risk of at least one of a low cardiac output syndrome (LOS), an acute kidney injury (AKI), a cerebral infarction, a mediastinitis, and an acute respiratory distress syndrome (ARDS) as the complication after the surgical operation.

10. The prediction display system according to claim 9, wherein the control section performs the prediction using the onset risk, and the display displays an extracorporeal circulation condition of the extracorporeal circulation apparatus, the extracorporeal circulation condition being likely to reduce the onset risk of the complication after the surgical operation.

11. The prediction display system according to claim 10, wherein the system is configured to suggest a change in flow rate and rotational speed of either a blood removing pump or a blood sending pump of the extracorporeal circulation apparatus.

12. The prediction display system according to claim 10, wherein the system determines whether or not there is a need for a hemoconcentrator circuit of the extracorporeal circulation apparatus on a basis of the onset risk of the complication after the surgical operation, and the display displays whether or not there is a need for the hemoconcentrator circuit.

13. The prediction display system according to claim 10, wherein the extracorporeal circulation condition is automatically changed based on the prediction.

14. The prediction display system according to claim 9, wherein an administration system for reducing the onset risk of the complication after the surgical operation is controlled based on the prediction.

15. The prediction display system according to claim 5, wherein the control section predicts the onset risk of the complication using at least one of an age, a sex, an address, a race, a family structure, an anamnesis, and a preoperative test value that affect a prognosis as characteristics of the patient.

16. The prediction display system according to claim 15, wherein the control section predicts the onset risk of the complication using at least one of an operative method, a lesion site, an operation time, an operator, an anesthesiologist, a dosage, a kind of medicine, an anesthesia induction time, and presence or absence of a blood transfusion as information regarding the surgical operation.

17. A prediction display system comprising:
a controller that that includes a control section that predicts an index of at least one of the number of ICU (intensive care unit) stay days, presence or absence of in-hospital death, and presence or absence of continuous renal replacement therapy (CRRT) introduction related to medical costs using: i) at least one of pH, pO2, Ht, Hb, DO2, and pCO2 as parameters of blood gas in data obtained during operation of an extracorporeal circulation apparatus; and ii) at least one of a pump flow rate, a pump rotational speed, a temperature, a blood circulation time, and an aorta interruption time as operating conditions of the extracorporeal circulation apparatus; and
a display that is connected to the control section and that displays the predicted index.

18. The prediction display system according to claim 17, wherein an onset risk of a main complication for a patient versus an other management item are two-dimensionally displayed on a matrix table in which one of the onset risk of the main complication and the other management item is an ordinate axis of the matrix table and the other of the onset risk of the main complication and the other management item is the abscissa axis of the matrix table, the control section predicts a transition of the patient's onset risk of the main complication versus and the other management item from one region to an other region in the matrix table during the ICU stay, and the other region to which the transition is made in the matrix table during the ICU stay is displayed on the display.

19. The prediction display system according to claim 18, wherein the matrix table two-dimensionally sets forth a group of high onset risks of the main complication and a group of low onset risks of the main complication, and a group of high probabilities of the number of ICU stay days, in-hospital death, and CRRT introduction related to medical cost and a group of low probabilities of the number of ICU stay days, in-hospital death, and CRRT introduction related to medical cost.

20. The prediction display system according to claim 18, wherein the matrix table has a group of high onset risks of the main complication and a group of low onset risks of the main complication, and a group of high risks of occurrence of a symptom that needs an action and a group of low risks of occurrence of the symptom.
